(19) United States
(12) Patent Application Publication
(10) Pub. No.: US 2011/0207770 A1
(43) Pub. Date: Aug. 25, 2011

(54) PHARMACEUTICAL PRODUCT COMPRISING A MUSCARINIC RECEPTOR ANTAGONIST AND A SECOND ACTIVE INGREDIENT

(75) Inventors: Barbara G. Avitabile, ESSEX (GB); Alan John Nadin, Essex (GB); Nicholas Charles Ray, Essex (GB)

(73) Assignees: ASTRAZENECA AB, SOEDERTAELJE (SE); PULMAGEN THERAPEUTICS (SYNERGY) LIMITED, HARLOW, ESSEX (GB)

(21) Appl. No.: 13/058,792

(22) PCT Filed: Aug. 11, 2009
(86) PCT No.: PCT/SE09/50924

§ 371 Date: May 10, 2011

(30) Foreign Application Priority Data
Aug. 12, 2008 (GB) 0814729.0

(51) Int. Cl. A61K 31/439; A61P 11/00

(52) U.S. Cl. 514/305

(57) ABSTRACT

The invention provides a pharmaceutical product, kit or composition comprising a first active ingredient which is a selected muscarinic receptor antagonist, and a second active ingredient which is selected from a phosphodiesterase inhibitor, a modulator of chemokine receptor function, an inhibitor of kinase function, a protease inhibitor, a steroidal glucocorticoid receptor agonist, a non-steroidal glucocorticoid receptor agonist and a purinoceptor antagonist, of use in the treatment of respiratory diseases such as chronic obstructive pulmonary disease and asthma.

The present invention relates to combinations of pharmaceutically active substances for use in the treatment of respiratory diseases, especially chronic obstructive pulmonary disease (COPD) and asthma.

The essential function of the lungs requires a fragile structure with enormous exposure to the environment, including pollutants, microbes, allergens, and carcinogens. Host factors, resulting from interactions of lifestyle choices and genetic composition, influence the response to this exposure. Damage or infection to the lungs can give rise to a wide range of diseases of the respiratory system (or respiratory diseases). A number of these diseases are of great public health importance. Respiratory diseases include Acute Lung Injury, Acute Respiratory Distress Syndrome (ARDS), occupational lung disease, lung cancer, tuberculosis, fibrosis, pneumoconiosis, pneumonia, emphysema, Chronic Obstructive Pulmonary Disease (COPD) and asthma.

Among the most common of the respiratory diseases is asthma. Asthma is generally defined as an inflammatory disorder of the airways with clinical symptoms arising from intermittent airflow obstruction. It is characterised clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

COPD is a term which refers to a large group of lung diseases which can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells. The two most important conditions covered by COPD are chronic bronchitis and emphysema.

Chronic bronchitis is a long-standing inflammation of the bronchi which causes increased production of mucous and other changes. The patients' symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

Therapeutic agents used in the treatment of respiratory diseases include muscarinic antagonists. Muscarinic receptors are a G-protein coupled receptor (GPCR) family having five family members M₁, M₂, M₃, M₄ and M₅. Of the five muscarinic subtypes, three (M₁, M₂ and M₃) are known to exert physiological effects on human lung tissue. Parasympathetic nerves are the main pathway for reflex bronchoconstriction in human airways and mediate airway tone by releasing acetylcholine onto muscarinic receptors. Airway tone is increased in patients with respiratory disorders such as asthma and chronic obstructive pulmonary disease (COPD), and for this reason muscarinic receptor antagonists have been developed for use in treating airway diseases. Muscarinic receptor antagonists, often called anticholinergics in clinical practice, have gained widespread acceptance as a first-line therapy for individuals with COPD, and their use has been extensively reviewed in the literature (e.g. Lee et al, Current Opinion in Pharmacology 2001, 1, 223-229).

Whilst treatment with a muscarinic antagonist can yield important benefits, the efficacy of these agents is often far from satisfactory. Moreover, in view of the complexity of respiratory diseases such as asthma and COPD, it is unlikely that any one mediator can satisfactorily treat the disease alone. Hence there is a pressing medical need for new therapies against respiratory diseases such as COPD and asthma, in particular for therapies with disease modifying potential.

The present invention provides a pharmaceutical product comprising, in combination, a first active ingredient which is a muscarinic antagonist selected from:
(R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X;
(R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X;
(R)-3-(3-Fluoro-4-methyl-phenoxy)-1-[3-(hydroxy-diphenyl-methyl)-isoxazol-5-ylmethyl]-1-azonia-bicyclo[2.2.2]octane X;
(R)-3-(3-Fluoro-phenylsulfanyl)-1-[3-(hydroxy-diphenyl-methyl)-isoxazol-5-ylmethyl]-1-azonia-bicyclo[2.2.2]octane X;
(R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X;
   wherein X represents a pharmaceutically acceptable anion of a mono or polyvalent acid, and a second active ingredient which is selected from
   i) a phosphodiesterase inhibitor,
   ii) a modulator of chemokine receptor function,
   iii) an inhibitor of kinase function,
   iv) a protease inhibitor,
   v) a steroidal glucocorticoid receptor agonist,
   vi) a non-steroidal glucocorticoid receptor agonist, and
   vii) a purinoceptor antagonist.

A beneficial therapeutic effect may be observed in the treatment of respiratory diseases if a muscarinic antagonist according to the present invention is used in combination with a second active ingredient as specified above. The beneficial effect may be observed when the two active substances are administered simultaneously (either in a single pharmaceutical preparation or via separate preparations), or sequentially or separately via separate pharmaceutical preparations.

The pharmaceutical product of the present invention may, for example, be a pharmaceutical composition comprising the first and second active ingredients in admixture. Alternatively, the pharmaceutical product may, for example, be a kit comprising a preparation of the first active ingredient and a preparation of the second active ingredient and, optionally, instructions for the simultaneous, sequential or separate administration of the preparations to a patient in need thereof.

The first active ingredient in the combination of the present invention is a muscarinic antagonist selected from:
(R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X;
(R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X;
(R)-3-(3-Fluoro-4-methyl-phenoxy)-1-[3-(hydroxy-diphenyl-methyl)-isoxazol-5-ylmethyl]-1-azonia-bicyclo[2.2.2]octane X;
(R)-3-(3-Fluoro-phenylsulfanyl)-1-[3-(hydroxy-diphenyl-methyl)-isoxazol-5-ylmethyl]-1-azonia-bicyclo[2.2.2]octane X;
(R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X;
   wherein X represents a pharmaceutically acceptable anion of a mono or polyvalent acid.

The muscarinic antagonists of the invention are selected members of a novel class of compound described in co-pending application PCT/GB2008/000519 (WO 2008/099186) which display high potency to the M3 receptor. The names of the muscarinic antagonists are IUPAC names generated by the Beilstein Autonom 2000 naming package, as supplied by MDL Information Systems Inc., based on the structures depicted in the examples, and stereochemistry assigned according to the Cahn-Ingold-Prelog system. For example, the name (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane, was generated from the structure:

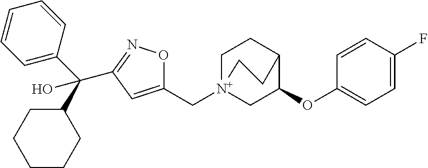

The muscarinic antagonists of the present invention comprise an anion X associated with the positive charge on the quaternary nitrogen atom. The anion X may be any pharmaceutically acceptable anion of a mono or polyvalent (e.g. bivalent) acid. In an embodiment of the invention X may be an anion of a mineral acid, for example chloride, bromide, iodide, sulfate, nitrate or phosphate; or an anion of a suitable organic acid, for example toluenesulfonate (tosylate), edisylate (ethane-1,2-disulfonate), isethionate (2-hydroxyethylsulfonate), lactate, oleic, maleate ((Z)-3-carboxy-acrylate), succinate (3-carboxy-propionate), malate ((S)-3-carboxy-2-hydroxy-propionate), p-acetamidobenzoateacetate, maleate, fumarate, citrate, oxalate, succinate, tartrate, methanesulphonate, p-toluenesulphonate, benzenesulphonate, napadisylate (naphthalene-1,5-disulphonate) (e.g. a heminapadisylate), 2,5-dichlorobenzenesulphonate, (xinafoate) 1-hydroxy-2-naphthoate or 1-hydroxynaphthalene-2-sulphonate.

In an embodiment of the invention, the muscarinic receptor antagonist is in the form of a bromide salt.

In an embodiment of the invention, the muscarinic receptor antagonist is selected from
(R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide;
(R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride;
(R)-3-(3-Fluoro-4-methyl-phenoxy)-1-[3-(hydroxy-diphenyl-methyl)-isoxazol-5-ylmethyl]-1-azonia-bicyclo[2.2.2]octane bromide;
(R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate;
(R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate;
(R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride; and
(R)-3-(3-Fluoro-phenylsulfanyl)-1-[3-(hydroxy-diphenyl-methyl)-isoxazol-5-ylmethyl]-1-azonia-bicyclo[2.2.2]octane bromide.

The second active ingredient of the present invention is selected from
i) a phosphodiesterase inhibitor,
ii) a modulator of chemokine receptor function,
iii) an inhibitor of kinase function,
iv) a protease inhibitor,
v) a steroidal glucocorticoid receptor agonist,
vi) a non-steroidal glucocorticoid receptor agonist, and
vii) a purinoceptor antagonist.

In an embodiment of the invention the second active ingredient is a phosphodiesterase inhibitor. Examples of a phosphodiesterase inhibitor that may be used according to this embodiment include a PDE4 inhibitor such as an inhibitor of the isoform PDE4D, a PDE3 inhibitor and a PDE5 inhibitor. Examples include the compounds
(Z)-3-(3,5-dichloro-4-pyridyl)-2-[4-(2-indanyloxy-5-methoxy-2-pyridyl]propenenitrile,
N-[9-amino-4-oxo-1-phenyl-3,4,6,7-tetrahydropyrrolo[3,2,1-jk][1,4]benzodiazepin-3(R)-yl]pyridine-3-carboxamide (CI-1044),
3-(benzyloxy)-1-(4-fluorobenzyl)-N-[3-(methylsulphonyl)phenyl]-1H-indole-2-carboxamide,
(1S-exo)-5-[3-(bicyclo[2.2.1]hept-2-yloxy)-4-methoxyphenyl]tetrahydro-2(1H)-pyrimidinone (Atizoram),
N-(3,5,dichloro-4-pyridinyl)-2-[1-(4-fluorobenzyl)-5-hydroxy-1H-indol-3-yl]-2-oxoacetamide (AWD-12-281),
β-[3-(cyclopentyloxy)-4-methoxyphenyl]-1,3-dihydro-1,3-dioxo-2H-isoindole-2-propanamide (CDC-801),
N-[9-methyl-4-oxo-1-phenyl-3,4,6,7-tetrahydropyrrolo[3,2,1-jk][1,4]benzodiazepin-3(R)-yl]pyridine-4-carboxamide (CI-1018),
cis-[4-cyano-4-(3-cyclopentyloxy-4-methoxyphenyl)cyclohexane-1-carboxylic acid (Cilomilast),
8-amino-1,3-bis(cyclopropylmethyl)xanthine (Cipamfylline),
N-(2,5-dichloro-3-pyridinyl)-8-methoxy-5-quinolinecarboxamide (D-4418),
5-(3,5-di-tert-butyl-4-hydroxybenzylidene)-2-iminothiazolidin-4-one (Darbufelone),
2-methyl-1-[2-(1-methylethyl)pyrazolo[1,5-a]pyridin-3-yl]-1-propanone (Ibudilast),
2-(2,4-dichlorophenylcarbonyl)-3-ureidobenzofuran-6-ylmethanesulphonate (Lirimilast), (−)-(R)-5-(4-methoxy-3-propoxyphenyl)-5-methyloxazolidin-2-one (Mesopram),
(−)-cis-9-ethoxy-8-methoxy-2-methyl-1,2,3,4,4a,10b-hexahydro-6-(4-diisopropylaminocarbonylphenyl)-benzo[c][1,6]naphthyridine (Pumafentrine), 3-(cyclopropylmethoxy)-N-(3,5-dichloro-4-pyridyl)-4-(difluoromethoxy)benzamide (Roflumilast),
the N-oxide of Roflumilast,
5,6-diethoxybenzo[b]thiophene-2-carboxylic acid (Tibenelast),
2,3,6,7-tetrahydro-2-(mesitylimino)-9,10-dimethoxy-3-methyl-4H-pyrimido[6,1-a]isoquinolin-4-one (trequinsin) and
3-[[3-(cyclopentyloxy)-4-methoxyphenyl]-methyl]-N-ethyl-8-(1-methylethyl)-3H-purine-6-amine (V-11294A).

In an embodiment of the invention the second active ingredient is a modulator of chemokine receptor function. Examples of a modulator of chemokine receptor function that may be used in this embodiment include a CCR3 receptor antagonist, a CCR4 receptor antagonist, a CCR5 receptor antagonist and a CCR8 receptor antagonist.

In an embodiment of the invention the second active ingredient is a CCR1 receptor antagonist.

In an embodiment of the invention, the second active ingredient is a CCR1 receptor antagonist selected from:
N-{2-[((2S)-3-{[1-(4-chlorobenzyl)piperidin-4-yl]amino}-2-hydroxy-2-methylpropyl)oxy]-4-hydroxyphenyl}acetamide;
N-{5-chloro-2-[((2S)-3-{[1-(4-chlorobenzyl)piperidin-4-yl]amino}-2-hydroxy-2-methylpropyl)oxy]-4-hydroxyphenyl}acetamide;
2-{2-chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid;
   or pharmaceutically acceptable salts thereof.

In another embodiment of the present invention, the second active ingredient is a salt of N-{2-[((2S)-3-{[1-(4-chlorobenzyl)piperidin-4-yl]amino}-2-hydroxy-2-methylpropyl)oxy]-4-hydroxyphenyl}acetamide or N-{5-Chloro-2-[((2S)-3-{[1-(4-chlorobenzyl)piperidin-4-yl]amino}-2-hydroxy-2-methylpropyl)oxy]-4-hydroxyphenyl}acetamide, for example hydrochloride, hydrobromide, phosphate, sulphate, acetate, ascorbate, benzoate, fumarate, hemifumarate, furoate, succinate, maleate, tartrate, citrate, oxalate, xinafoate, methanesulphonate or p-toluenesulphonate salt.

In another embodiment of the present invention, the second active ingredient is a benzoate, furoate or hemifumarate salt of N-{2-[((2S)-3-{[1-(4-chlorobenzyl)piperidin-4-yl]amino}-2-hydroxy-2-methylpropyl)oxy]-4-hydroxyphenyl}acetamide, as described in PCT/SE2006/000920, PCT/SE2006/000921 and PCT/SE2006/000922 (WO2007/015666, WO2007/015667 and WO2007/015668).

In another embodiment of the present invention, the second active ingredient is the hemifumarate, furoate, benzoate, 2-fluorobenzoate or 2,6-difluorobenzoate salt of N-{5-Chloro-2-[((2S)-3-{[1-(4-chlorobenzyl)piperidin-4-yl]amino}-2-hydroxy-2-methylpropyl)oxy]-4-hydroxyphenyl}acetamide.

In an embodiment of the present invention the second active ingredient is 2-{2-chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof. 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid may be prepared by methods according or analogous to those described in PCT/SE2007/000694 (WO2008/010765).

In an embodiment of the present invention the second active ingredient is N-{5-chloro-2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof. N-{5-chloro-2-[((2S)-3-{[1-(4-chlorobenzyl)piperidin-4-yl]amino}-2-hydroxy-2-methylpropyl)oxy]-4-hydroxyphenyl}acetamide may be prepared by methods according or analogous to those described in WO2007/015664.

In an embodiment of the invention, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X, wherein X represents a pharmaceutically acceptable anion of a mono or polyvalent acid, and the second active ingredient is N-{2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof (e.g. benzoate, hemifumarate or furoate). In one aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate.

In an embodiment of the invention, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X, wherein X represents a pharmaceutically acceptable anion of a mono or polyvalent acid, and the second active ingredient is N-{2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof (e.g. benzoate, hemifumarate or furoate). In one aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate.

In an embodiment of the invention, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X, wherein X represents a pharmaceutically acceptable anion of a mono or polyvalent acid, and the second active ingredient is N-{2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof (e.g. benzoate, hemifumarate or furoate). In one aspect of this embodiment, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide.

In an embodiment of the invention, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the second active ingredient is N-{2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof (e.g. benzoate, hemifumarate or furoate). In one aspect of this embodiment, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the second active ingredient is N-{2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof (e.g. benzoate, hemifumarate or furoate). In one aspect of this embodiment, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X, wherein X represents a pharmaceutically acceptable anion of a mono or polyvalent acid, and the second active ingredient is N-{5-chloro-2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof (e.g. benzoate, hemifumarate or furoate). In one aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate.

In an embodiment of the invention, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X, wherein X represents a pharmaceutically acceptable anion of a mono or polyvalent acid, and the second active ingredient is N-{5-chloro-2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof (e.g. benzoate, hemifumarate or furoate). In one aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate.

In an embodiment of the invention, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X, wherein X represents a pharmaceutically acceptable anion of a mono or polyvalent acid, and the second active ingredient is N-{5-chloro-2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof (e.g. benzoate, hemifumarate or furoate). In one aspect of this embodiment, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide.

In an embodiment of the invention, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the second active ingredient is N-{5-chloro-2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof (e.g. benzoate, hemifumarate or furoate). In one aspect of this embodiment, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the second active ingredient is N-{5-chloro-2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof (e.g. benzoate, hemifumarate or furoate). In one aspect of this embodiment, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X, wherein X represents a pharmaceutically acceptable anion of a mono or polyvalent acid, and the second active ingredient is 2-{2-Chloro-5-{[(2S)-3-(5-chloro-1H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof. In one aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate.

In an embodiment of the invention, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X, wherein X represents a pharmaceutically acceptable anion of a mono or polyvalent acid, and the second active ingredient is 2-{2-Chloro-5-{[(2S)-3-(5-chloro-1H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof. In one aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate.

In an embodiment of the invention, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X, wherein X represents a pharmaceutically acceptable anion of a mono or polyvalent acid, and the second active ingredient is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof. In one aspect of this embodiment, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide.

In an embodiment of the invention, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the second active ingredient is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof. In one aspect of this embodiment, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the second active ingredient is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof. In one aspect of this embodiment, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide.

In an embodiment of the invention the second active ingredient is an inhibitor of kinase function. Examples of an inhibitor of kinase function that may be used in this embodiment include a p38 kinase inhibitor and an IKK inhibitor.

In an embodiment of the invention the second active ingredient is a protease inhibitor. Examples of a protease inhibitor that may be used in this embodiment include an inhibitor of neutrophil elastase or an inhibitor of MMP12.

In an embodiment of the invention the second active ingredient is a steroidal glucocorticoid receptor agonist. Examples of a steroidal glucocorticoid receptor agonist that may be used in this embodiment include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (as e.g. dicloacetate), triamcinolone (e.g. as acetonide), flunisolide, zoticasone, flumoxonide, rofleponide, butixocort (e.g. as propionate ester), prednisolone, prednisone, tipredane, steroid esters e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters according to DE 4129535, steroids according to WO 2002/00679, WO 2005/041980, or steroids GSK 870086, GSK 685698 and GSK 799943.

In an embodiment of the invention, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X, wherein X represents a pharmaceutically acceptable anion of a mono or polyvalent acid, and the second active ingredient is budesonide. In one aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate.

In an embodiment of the invention, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X, wherein X represents a pharmaceutically acceptable anion of a mono or polyvalent acid, and the second active ingredient is budesonide. In one aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride. In another aspect of this embodiment, the muscarinic receptor antagonist is (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate.

In an embodiment of the invention, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X, wherein X represents a pharmaceutically acceptable anion of a mono or polyvalent acid, and the second active ingredient is budesonide. In one aspect of this embodiment, the muscarinic receptor antagonist is (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide.

In an embodiment of the invention, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the second active ingredient is budesonide. In one aspect of this embodiment, the muscarinic receptor antagonist is (R)-3-(3-Fluoro-phenylsulfanyl)-1-[3-(hydroxy-diphenyl-methyl)-isoxazol-5-ylmethyl]-1-azonia-bicyclo[2.2.2]octane bromide.

In an embodiment of the invention, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane X, wherein X represents a pharmaceutically acceptable anion of a mono or polyvalent acid, and the second active ingredient is budesonide. In one aspect of this embodiment, the muscarinic receptor antagonist is (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide.

In an embodiment of the invention the second active ingredient is a non-steroidal glucocorticoid receptor agonist. Examples of a modulator of a non-steroidal glucocorticoid receptor agonist that may be used in this embodiment include selective non-steroidal glucocorticoid receptor agonists. Non-steroidal glucocorticoid receptor agonists are described for example in WO2006/046916 and U.S. Pat. No. 6,323,199.

In an embodiment of the invention the second active ingredient is a purinoceptor antagonist, for example a P2X₇ receptor antagonist. Examples of P2X₇ receptor antagonists are described in WO00/61569, WO01/44170, WO01/94338, WO03/041707, WO03/080579, WO04/106305, WO05/009968, WO06/025784 and WO06/059945.

The combination of the present invention may provide a beneficial therapeutic effect in the treatment of respiratory diseases. Examples of such possible effects include improvements in one or more of the following parameters: reducing inflammatory cell influx into the lung, mild and severe exacerbations, FEV₁ (forced expiratory volume in one second), vital capacity (VC), peak expiratory flow (PEF), symptom scores and Quality of Life.

The muscarinic antagonist (first active ingredient) and second active ingredient of the present invention may be administered simultaneously, sequentially or separately to treat respiratory diseases. By sequential it is meant that the active ingredients are administered, in any order, one immediately after the other. They may still have the desired effect if they are administered separately, but when administered in this manner they will generally be administered less than 4 hours apart, more conveniently less than two hours apart, more conveniently less than 30 minutes apart and most conveniently less than 10 minutes apart.

The active ingredients of the present invention may be administered by oral or parenteral (e.g. intravenous, subcutaneous, intramuscular or intraarticular) administration using conventional systemic dosage forms, such as tablets, capsules, pills, powders, aqueous or oily solutions or suspensions, emulsions and sterile injectable aqueous or oily solutions or suspensions. The active ingredients may also be administered topically (to the lung and/or airways) in the form of solutions, suspensions, aerosols and dry powder. These dosage forms will usually include one or more pharmaceutically acceptable ingredients which may be selected, for example, from adjuvants, carriers, binders, lubricants, diluents, stabilising agents, buffering agents, emulsifying agents, viscosity-regulating agents, surfactants, preservatives, flavourings and colorants. As will be understood by those skilled in the art, the most appropriate method of administering the active ingredients is dependent on a number of factors.

In one embodiment of the present invention the active ingredients are administered via separate pharmaceutical preparations. Therefore, in one aspect, the present invention provides a kit comprising a preparation of a first active ingredient which is a muscarinic antagonist according to the present invention, and a preparation of a second active ingredient, and optionally instructions for the simultaneous, sequential or separate administration of the preparations to a patient in need thereof.

In another embodiment the active ingredients may be administered via a single pharmaceutical composition. Therefore, the present invention further provides a pharmaceutical composition comprising, in admixture, a first active ingredient, which is a muscarinic antagonist according to the present invention, and a second active ingredient, as defined above.

The pharmaceutical compositions of the present invention may be prepared by mixing the muscarinic antagonist (first active ingredient) with the second active ingredient and a pharmaceutically acceptable adjuvant, diluent or carrier. Therefore, in a further aspect of the present invention there is provided a process for the preparation of a pharmaceutical composition, which comprises mixing a muscarinic antagonist according to the present invention with a second active ingredient according to the present invention and a pharmaceutically acceptable adjuvant, diluent or carrier.

It will be understood that the therapeutic dose of each active ingredient administered in accordance with the present invention will vary depending upon the particular active ingredient employed, the mode by which the active ingredient is to be administered, and the condition or disorder to be treated.

In one embodiment of the present invention, the muscarinic antagonist (first active ingredient) according to the present invention is administered via inhalation. When administered via inhalation the dose of the muscarinic antagonist according to the present invention will generally be in the range of from 0.1 microgram (μg) to 5000 μg, 0.1 to 1000 μg, 0.1 to 500 μg, 0.1 to 100 μg, 0.1 to 50 μg, 0.1 to 5 μg, 5 to 5000 μg, 5 to 1000 μg, 5 to 500 μg, 5 to 100 μg, 5 to 50 μg, 5 to 10 μg, 10 to 5000 μg, 10 to 1000 μg, 10 to 500 μg, 10 to 100 μg, 10 to 50 μg, 20 to 5000 μg, 20 to 1000 μg, 20 to 500 μg, 20 to 100 μg, 20 to 50 μg, 50 to 5000 μg, 50 to 1000 μg, 50 to 500 μg, 50 to 100 μg, 100 to 5000 μg, 100 to 1000 μg or 100 to 500 μg. The dose will generally be administered from 1 to 4 times a day, conveniently once or twice a day, and most conveniently once a day.

In one embodiment of the present invention the second active ingredient of the present invention may conveniently be administered by inhalation. When administered via inhalation the dose of the second active ingredient will generally be in the range of from 0.1 to 50 μg, 0.1 to 40 μg, 0.1 to 30 μg, 0.1 to 20 μg, 0.1 to 10 μg, 5 to 10 μg, 5 to 50 μg, 5 to 40 μg, 5 to 30 μg, 5 to 20 μg, 5 to 10 μg, 10 to 50 μg, 10 to 40 μg 10 to 30 μg, or 10 to 20 μg. The dose will generally be administered from 1 to 4 times a day, conveniently once or twice a day, and most conveniently once a day.

In another embodiment of the present invention, the second active ingredient is administered orally. Oral administration of the second active ingredient may for example be used in a pharmaceutical product or kit wherein the other active ingredient(s) are administered by inhalation. When administered orally, satisfactory results will generally be obtained when the dose of the second active ingredient is in the range of from 5 to 1000 milligram (mg), 5 to 800 mg, 5 to 600 mg, 5 to 500 mg, 5 to 400 mg, 5 to 300 mg, 5 to 200 mg, 5 to 100 mg, 5 to 50 mg, 20 to 1000 mg, 20 to 800 mg, 20 to 600 mg, 20 to 500 mg, 20 to 400 mg, 20 to 300 mg, 20 to 200 mg, 20 to 100 mg, 20 to 50 mg, 50 to 1000 mg, 50 to 800 mg, 50 to 600 mg, 50 to 500 mg, 50 to 400 mg, 50 to 300 mg, 50 to 200 mg, 50 to 100 mg, 100 to 1000 mg, 100 to 800 mg, 100 to 600 mg, 100 to 500 mg, 100 to 400 mg, 100 to 300 mg, or 100 to 200 mg. The dose will generally be administered from 1 to 4 times a day, conveniently once or twice a day, and most conveniently once a day.

In one embodiment, the present invention provides a pharmaceutical product comprising, in combination, a first active ingredient which is a muscarinic antagonist, and a second active ingredient, as defined herein above, wherein each active ingredient is formulated for inhaled administration.

In another embodiment of the present invention, the first active ingredient, which is a muscarinic antagonist, may be formulated for oral administration and the second active ingredient(s), as defined herein above, may be formulated for inhaled administration.

In yet another embodiment of the present invention, the first active ingredient, which is a muscarinic antagonist, may be formulated for inhaled administration and the second active ingredient(s), as defined herein above, may be formulated for oral administration.

In yet a further embodiment of the present invention, the first active ingredient, which is a muscarinic antagonist, and the second active ingredient(s), as defined herein above, wherein each active ingredient is formulated for oral administration.

In an embodiment the pharmaceutical preparations of active ingredients may be administered simultaneously.

In an embodiment the different pharmaceutical preparations of active ingredients may be administered sequentially.

In an embodiment the different pharmaceutical preparations of active ingredients may be administered separately.

The active ingredients of the present invention are conveniently administered via inhalation (e.g. topically to the lung and/or airways) in the form of solutions, suspensions, aerosols and dry powder formulations. For example metered dose inhaler devices may be used to administer the active ingredients, dispersed in a suitable propellant and with or without additional excipients such as ethanol, surfactants, lubricants or stabilising agents. Suitable propellants include hydrocarbon, chlorofluorocarbon and hydrofluoroalkane (e.g. heptafluoroalkane) propellants, or mixtures of any such propellants. Preferred propellants are P134a and P227, each of which may be used alone or in combination with other propellants and/or surfactant and/or other excipients. Nebulised aqueous suspensions or, preferably, solutions may also be employed, with or without a suitable pH and/or tonicity adjustment, either as a unit-dose or multi-dose.

Dry powders and pressurized HFA aerosols of the active ingredients may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

The combination of the present invention is useful in the treatment or prevention of respiratory-tract disorders such as chronic obstructive pulmonary disease (COPD), chronic bronchitis of all types (including dyspnea associated therewith), asthma (allergic and non-allergic; ‘wheezy-infant syndrome’), adult/acute respiratory distress syndrome (ARDS), chronic respiratory obstruction, bronchial hyperactivity, pulmonary fibrosis, pulmonary emphysema, and allergic rhinitis, exacerbation of airway hyperreactivity consequent to other drug therapy, particularly other inhaled drug therapy or pneumoconiosis (for example aluminosis, anthracosis, asbestosis, chalicosis, ptilosis, siderosis, silicosis, tabacosis and byssinosis).

Dry powder inhalers may be used to administer the active ingredients, alone or in combination with a pharmaceutically acceptable carrier, in the later case either as a finely divided powder or as an ordered mixture. The dry powder inhaler may be single dose or multi-dose and may utilise a dry powder or a powder-containing capsule.

Metered dose inhaler, nebuliser and dry powder inhaler devices are well known and a variety of such devices are available.

The present invention further provides a pharmaceutical product, kit or pharmaceutical composition according to the invention for simultaneous, sequential or separate use in therapy.

The present invention further provides the use of a pharmaceutical product, kit or pharmaceutical composition according to the invention in the manufacture of a medicament for the treatment of a respiratory disease, in particular chronic obstructive pulmonary disease or asthma.

The present invention further provides a pharmaceutical product, kit or pharmaceutical composition according to the invention for use in the treatment of a respiratory disease, in particular chronic obstructive pulmonary disease or asthma.

The present invention still further provides a method of treating a respiratory disease which comprises simultaneously, sequentially or separately administering:

(a) a (therapeutically effective) dose of a first active ingredient which is a muscarinic antagonist according to the present invention; and
(b) a (therapeutically effective) dose of a second active according to the present invention;
to a patient in need thereof.

In the context of the present specification, the term “therapy” also includes “prophylaxis” unless there are specific indications to the contrary. The terms “therapeutic” and “therapeutically” should be construed accordingly. Prophylaxis is expected to be particularly relevant to the treatment of persons who have suffered a previous episode of, or are otherwise considered to be at increased risk of, the condition or disorder in question. Persons at risk of developing a particular condition or disorder generally include those having a family history of the condition or disorder, or those who have been identified by genetic testing or screening to be particularly susceptible to developing the condition or disorder.

The term “disease, unless stated otherwise, has the same meaning as the terms “condition” and “disorder” and are used interchangeably throughout the description and claims.

The term “agent” and “active ingredient” means the compounds comprised in the combination of the present invention, e.g. a muscarinic antagonist or a CCR1 antagonist.

The pharmaceutical product, kit or composition of the present invention may optionally comprise a third active ingredient which third active ingredient is a substance suitable for use in the treatment of respiratory diseases.

Examples of third active ingredients that may be incorporated into the present invention include those listed herein above as second active ingredients (i.e. a phosphodiesterase inhibitor, a modulator of chemokine receptor function, an inhibitor of kinase function, a protease inhibitor, a steroidal glucocorticoid receptor agonist, a non-steroidal glucocorticoid receptor agonist or a purinoceptor antagonist) it being recognised that they may be utilised as third active ingredients in embodiments where they have not been utilised as the second active ingredient.

In one embodiment of the invention, the third active ingredient is a β₂-adrenoceptor agonist. The β₂-adrenoceptor agonist may be any compound or substance capable of stimulating the β₂-receptors and acting as a bronchodilator. Examples of β₂-adrenoceptor agonists that may be employed in the present invention include formoterol. The chemical name for formoterol is N-[2-hydroxy-5-[(1)-1-hydroxy-2-[[(1)-2-(4-methoxyphenyl)-1-methylethyl]amino]ethyl]phenyl]-formamide. The preparation of formoterol is described, for example, in WO 92/05147. In one aspect of this embodiment, the β₂-adrenoceptor agonist is formoterol fumarate. It will be understood that the invention encompasses the use of all optical isomers of formoterol and mixtures thereof including racemates. Thus for example, the term formoterol encompasses N-[2-hydroxy-5-[(1R)-1-hydroxy-2-[[(1R)-2-(4-methoxyphenyl)-1-methylethyl]amino]ethyl]phenyl]-formamide, N-[2-hydroxy-5-[(1S)-1-hydroxy-2-[[(1S)-2-(4-methoxyphenyl)-1-methylethyl]amino]ethyl]phenyl]-formamide and a mixture of such enantiomers, including a racemate.

In an alternative embodiment of the present invention, the pharmaceutical product, kit or pharmaceutical composition does not contain a β₂-adrenoceptor agonist.

The invention is illustrated by the following non-limiting Examples. In the Examples the following Figures are presented:

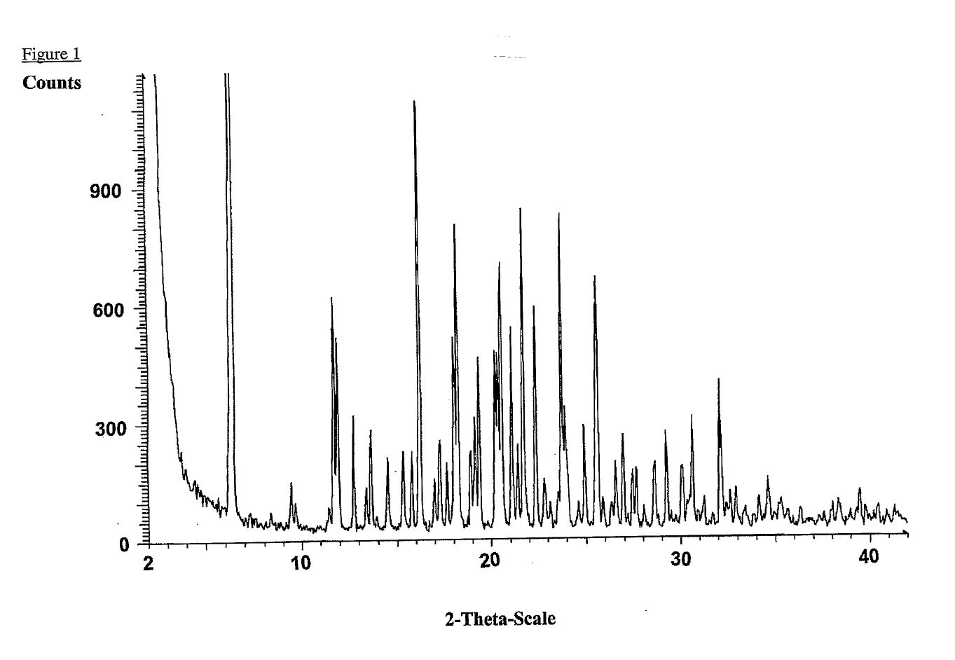

FIG. 1: X-ray powder diffraction pattern of muscarinic antagonist (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane; benzenesulfonate (Example 2).

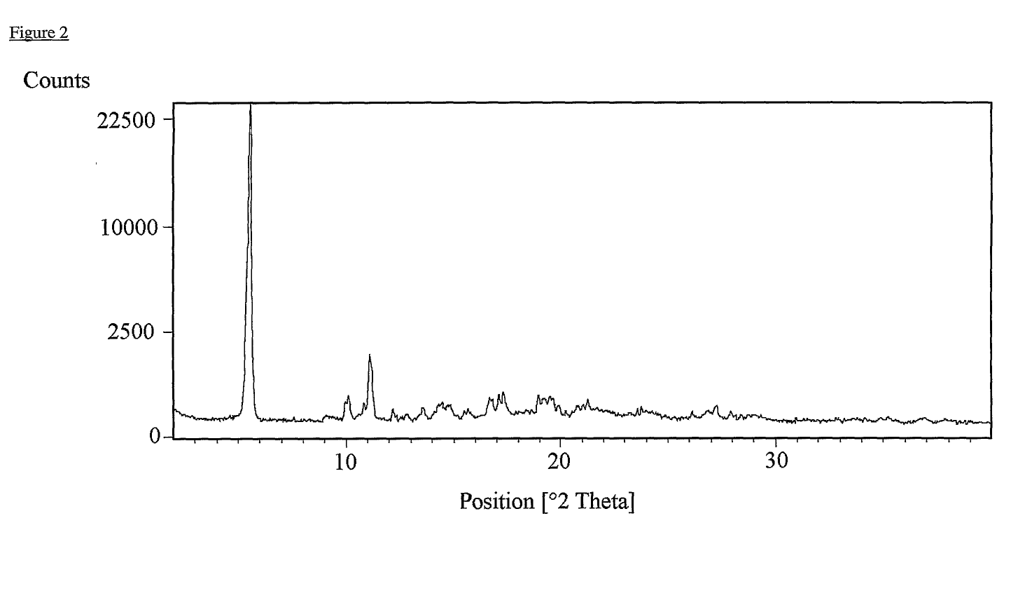

FIG. 2: X-ray powder diffraction pattern of muscarinic antagonist (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride (Example 3).

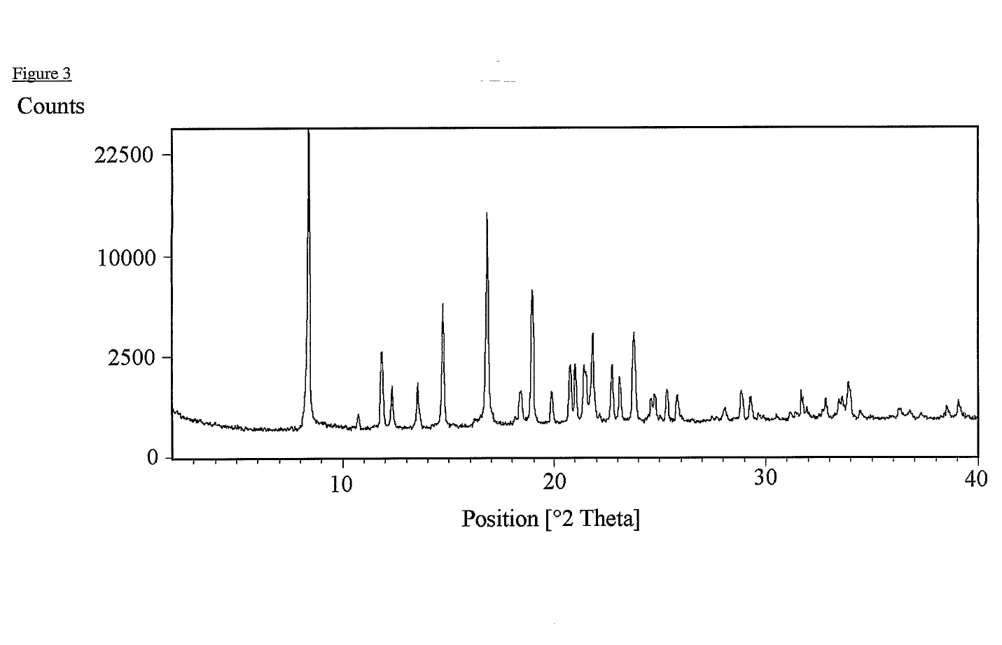

FIG. 3: X-ray powder diffraction pattern of muscarinic antagonist (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (Example 4).

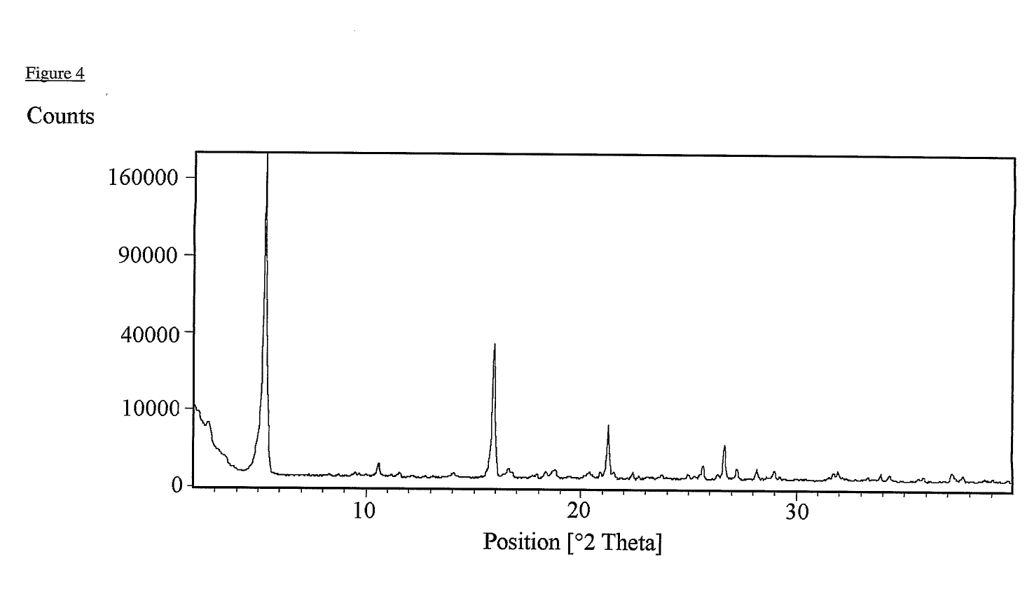

FIG. 4: X-ray powder diffraction pattern of muscarinic antagonist (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide (Example 5).

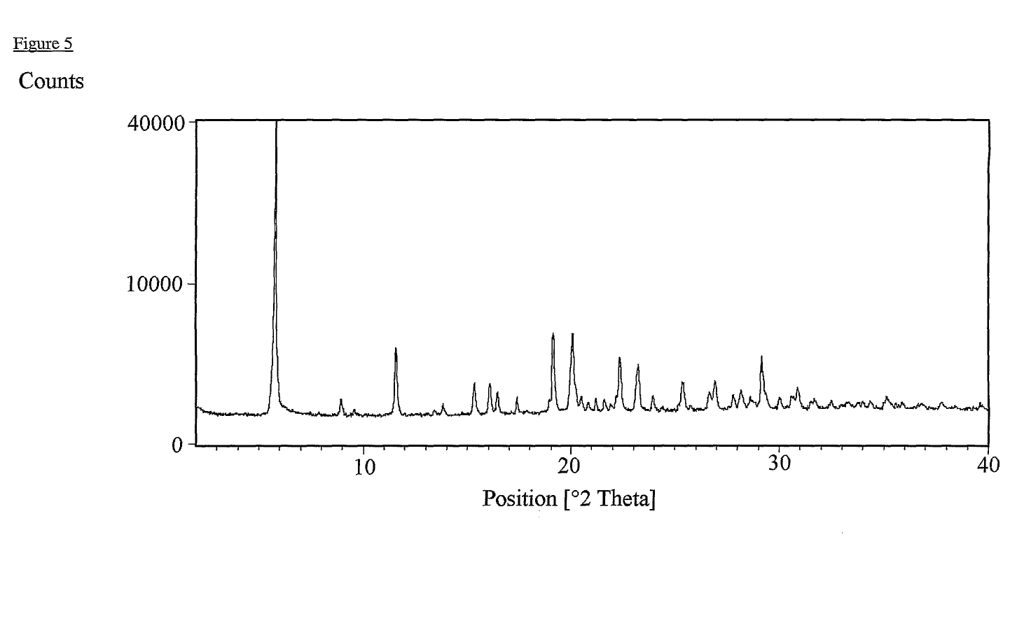

FIG. 5: X-ray powder diffraction pattern of muscarinic antagonist (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide (Example 7).

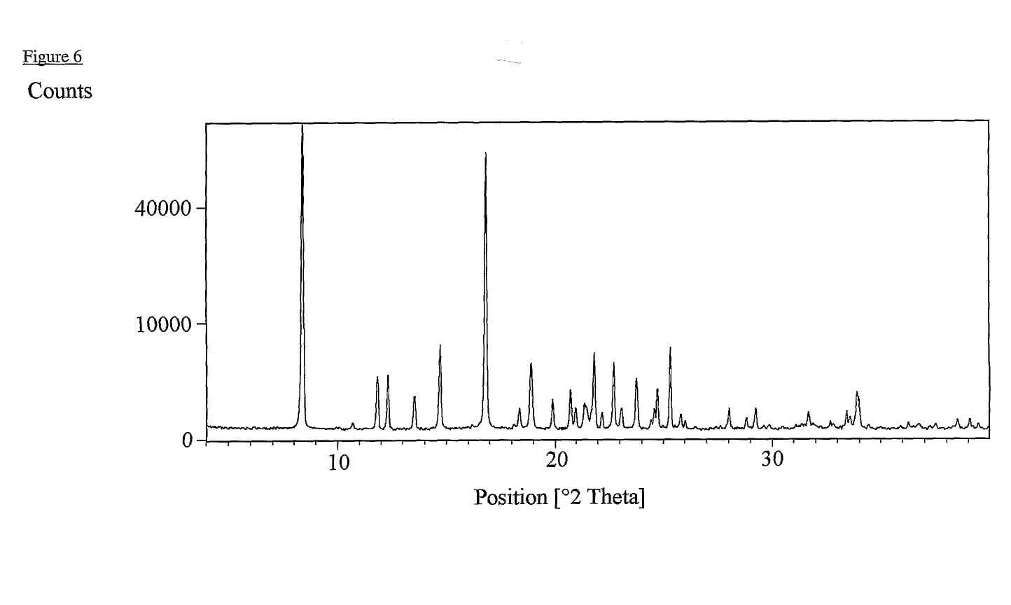

FIG. 6: X-ray powder diffraction pattern of muscarinic antagonist (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (Example 8)

### PREPARATION OF MUSCARINIC ANTAGONISTS

Muscarinic antagonists according to the present invention may be prepared as follows.

Alternative salts to those described herein may be prepared by conventional chemistry using methods analogous to those described.

### General Experimental Details for Preparation of Muscarinic Antagonists

Unless otherwise stated the following general conditions were used in the preparation of the Muscarinic Antagonists

All reactions were carried out under an atmosphere of nitrogen unless specified otherwise. NMR spectra were obtained on a Varian Unity Inova 400 spectrometer with a 5 mm inverse detection triple resonance probe operating at 400 MHz or on a Bruker Avance DRX 400 spectrometer with a 5 mm inverse detection triple resonance TXI probe operating at 400 MHz or on a Bruker Avance DPX 300 spectrometer with a standard 5 mm dual frequency probe operating at 300 MHz. Shifts are given in ppm relative to tetramethylsilane. Where products were purified by column chromatography, ‘flash silica’ refers to silica gel for chromatography, 0.035 to 0.070 mm (220 to 440 mesh) (e.g. Fluka silica gel 60), and an applied pressure of nitrogen up to 10 p.s.i accelerated column elution or use of the semi-automated CombiFlash® Companion purification system or by manual elution of Biotage® Isolute Flash Si II cartridges under reduced pressure or by use of the Biotage® SP1 semi-automated system. All solvents and commercial reagents were used as received. SCX chromatography was performed on Biotage® Isolute SCX or SCX-2 pre-packed cartridges.

The Liquid Chromatography Mass Spectroscopy (LCMS) methods referred to are described below:

### Method 1

Waters Micromass ZQ2000 with a C18-reverse-phase column (100×3.0 mm Higgins Clipeus with 5 μm particle size), elution with A: water+0.1% formic acid; B: acetonitrile+0.1% formic acid. Gradient:

| Gradient - Time | flow mL/min | % A | % B |
|---|---|---|---|
| 0.00 | 1.0 | 95 | 5 |
| 1.00 | 1.0 | 95 | 5 |
| 15.00 | 1.0 | 5 | 95 |
| 20.00 | 1.0 | 5 | 95 |
| 22.00 | 1.0 | 95 | 5 |
| 25.00 | 1.0 | 95 | 5 |

Detection—MS, ELS, UV (100 μl split to MS with in-line UV detector) MS ionisation method—Electrospray (positive ion)

### Method 2

Waters Platform LC Quadrupole mass spectrometer with a C18-reverse-phase column (30×4.6 mm Phenomenex Luna 3 μm particle size), elution with A: water+0.1% formic acid; B: acetonitrile+0.1% formic acid. Gradient:

| Gradient - Time | flow mL/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection—MS, ELS, UV (200 μl split to MS with in-line UV detector) MS ionisation method—Electrospray (positive and negative ion).

Abbreviations used in the experimental section: AIBN=2,2′-azobis(2-methylpropionitrile); DCM=dichloromethane; DMF=dimethylformamide; DMSO=dimethyl sulfoxide; IMS=industrial methylated spirit; LCMS=Liquid Chromatography-Mass Spectrometry; NBS=N-bromosuccinimide; RT=room temperature; Rt=retention time; TFA=trifluoroacetic acid; THF=tetrahydrofuran; SCX=strong cation exchange chromatography.

### For the Analysis of the Crystalline Form of Example 2:

Differential Scanning calorimetry (DSC) measurements were performed on a Mettler Toledo DSC823e equipped with a Mettler Toledo TS0801RO sample robot and automated sample carousel. Samples were prepared in 40 μl aluminium pans, the sample lids were automatically pierced by the robot and the analysis undertaken between 30 and 250° C. at 10° C./min. Typically, 1-3 mg of sample was used for analysis and the analysis was performed under dry nitrogen purged at 50 mlmin⁻¹. The instrument was calibrated for energy and temperature using certified indium.

Thermogravimmetric analysis (TGA) analysis was determined using a Mettler Toledo thermogravimetric analyser (TGA851e) equipped with a TS0801 RO sample robot and automated sample carousel. Each pan lid was pierced manually before analysis and run between 30 and 400° C. at 10° C./min. Typically, 1-3 mg of sample was used for analysis. A nitrogen purge at 60 mlmin⁻¹ was maintained over the sample during analysis. The instrument was calibrated for temperature.

Powder X-ray diffraction (PXRD) data were collected on a Bruker AXS C2 GADDS diffractometer using Cu K_{α} radiation (40 kV, 40 mA), an automated XYZ stage, a laser video microscope for auto-sample positioning and a HiStar 2 dimensional area detector. The X-ray optics consisted of a single Göbel multilayer mirror coupled with a pinhole collimator of 0.3 mm. The beam divergence, i.e. the effective size of the X-ray beam on the sample, was approximately 4 mm. A θ-θ continuous scan mode was employed with a sample to detector distance which gave an effective 2θ range of 3.2° to 42.7°. Typically the sample was exposed to the X-ray beam for 120 seconds. Samples were prepared as flat plate specimens using material as received without grinding. Approximately 1-2 mg of the sample was lightly pressed on a glass slide to obtain a flat surface.

Dynamic vapour sorption (DVS) analysis was performed on a Surface Measurement systems (SMS) DVS-Intrinsic moisture sorption analyser. The instrument was controlled by SMS Analysis Suite software (DVS-Intrinsic Control v1.0.0.30). Analysis of the data was performed using Microsoft Excel 2007 together DVS Standard Analysis Suite (v6.0.0.7). Sample temperature was maintained at 25° C. and the sample humidity was obtained by mixing streams of wet and dry nitrogen at a total flow rate of 200 mlmin⁻¹. The relative humidity was measured using a calibrated Rotronic probe (dynamic range 1-100% Relative Humidity (RH)) located close to the sample. The weight change of the sample as a function of % RH was constantly monitored by the microbalance (accuracy ±0.005 mg). Typically a PXRD would be run prior to analysis. 20 mg of sample was then placed in a tared stainless steel mesh basket under ambient conditions. The sample was loaded and unloaded at 40% RH and 25° C. (typical room conditions) and the sample subjected to a graduated DVS regime over 2 cycles using the parameters shown in Table 1. A DVS isotherm was calculated from this data and a final PXRD was performed after analysis to check for change in solid state form.

TABLE 1
Method parameters for DVS experiment

| Parameter | Setting |
|---|---|
| Sorption - cycle 1 (% RH) | 40-90 |
| Desorption - cycle 1 (% RH) | 90-0 |
| Sorption - cycle 2 (% RH) | 0-90 |
| Desorption - cycle 2 (% RH) | 90-0 |
| Sorption - cycle 3 (% RH) | 0-40 |
| Intervals (% RH) | 10 |
| dmdt (% min⁻¹) | 0.002 |
| Sample temperature (° C.) | 25 |

### For the Analysis of the Crystalline Forms of Examples 3, 4, 5, 7 and 8:

X-Ray Powder Diffraction (XRPD)-PANalytical X'Pert machine in 2Ø-Ø configuration or a PANalytical Cubix machine in Ø-Ø configuration over the scan range 2° to 40° 2Ø with 100-second exposure per 0.02° increment. The X-rays were generated by a copper long-fine focus tube operated at 45 kV and 40 mA. The wavelength of the copper X-rays was 1.5418 Å. The Data was collected on zero background holders on which ˜2 mg of the compound was placed. The holder was made from a single crystal of silicon, which had been cut along a non-diffracting plane and then polished on an optically flat finish. The X-rays incident upon this surface were negated by Bragg extinction.

Differential Scanning calorimetry (DSC) thermograms were measured using a TA Q1000 Differential Scanning calorimeter, with aluminium pans and pierced lids. The sample weights varied between 0.5 to 5 mg. The procedure was carried out under a flow of nitrogen gas (50 ml/min) and the temperature studied from 25 to 300° C. at a constant rate of temperature increase of 10° C. per minute.

Thermogravimetric Vapour Sorption (TGA) thermograms were measured using a TA Q500 Thermogravimetric Analyser, with platinum pans. The sample weights varied between 1 and 5 mg. The procedure was carried out under a flow of nitrogen gas (60 ml/min) and the temperature studied from Room Temperature to 300° C. at a constant rate of temperature increase of 10° C. per minute.

Gravimetric Vapour Sorption (GVS) profiles were measured using a Surface Measurements Systems Dynamic Vapour Sorption DVS-1 or a DVS Advantage instrument. The solid sample ca. 1-5 mg was placed into a glass vessel and the weight of the sample was recorded during a dual cycle step method (40 to 90 to 0 to 90 to 0% relative humidity (RH), in steps of 10% RH).

### INTERMEDIATE 1

### (R)-3-(3-Fluoro-phenoxy)-1-aza-bicyclo[2.2.2]octane

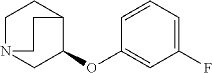

A solution of (R)-1-aza-bicyclo[2.2.2]octan-3-ol (1.25 g), CuI (93.1 mg), 1,10-phenanthroline (176 mg), Cs₂CO₃ (3.19 g) and 3-fluoro-iodo-benzene (1.11 g) in toluene (2.5 mL) was heated at 100° C. for 20 h. The reaction mixture was cooled, diluted with ethyl acetate and filtered through Celite. The insoluble material was washed several times with ethyl acetate. The filtrate was washed with 5% copper sulphate solution, water, dried (MgSO₄), filtered and evaporated in vacuo. Purification by SCX gave (R)-3-(3-fluoro-phenoxy)-1-aza-bicyclo[2.2.2]octane (490 mg, 45%) as a brown oil. LCMS (Method 2, Rt 2.09 min). MH⁺=222.

Intermediates 2-3 were prepared from (R)-1-aza-bicyclo[2.2.2]octan-3-ol and the appropriate aryl iodide by analogy with the procedure described for Intermediate 1. Data for Intermediates 2-3:

| | | LCMS (Method, |
|---|---|---|
| Intermediate | | Retention Time, |
| Number | Structure | MH+) |
| 2 | 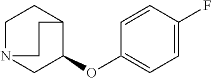 | 2, 2.05 min, 222 |
| 3 | 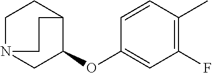 | 2, 2.21 min, 236 |

### INTERMEDIATE 4

### (R)-3-(3-Fluoro-phenylsulfanyl)-1-aza-bicyclo[2.2.2]octane

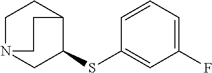

(R)-3-(3-Fluoro-phenylsulfanyl)-1-aza-bicyclo[2.2.2]octane was prepared from 3-fluorothiophenol as follows: A solution of 3-fluorothiophenol (5 g) in DMF (5 ml) was added slowly to a suspension of NaH (1.56 g of 60% dispersion in mineral oil) in DMF (40 mL) at room temperature. After 30 min, a solution of methanesulfonic acid (S)-(1-aza-bicyclo[2.2.2]oct-3-yl) ester (5.3 g) (J. Med. Chem., 1992, 35, 2392-2406) in DMF (5 mL) was added to the mixture dropwise and the reaction mixture was heated at 70° C. overnight. The reaction mixture was partitioned between ethyl acetate and 1 N NaOH solution. The layers were separated and the aqueous phase was extracted with ethyl acetate. The combined organic layers were washed with brine, dried (MgSO₄), filtered and evaporated in vacuo. Purification by SCX chromatography gave (R)-3-(3-fluoro-phenylsulfanyl)-1-aza-bicyclo[2.2.2]octane (4.5 g, 73%). Data for Intermediate 4: NMR (300 MHz, MeOD): 7.33 (1H, td, J=8.04, 6.01 Hz), 7.22-7.13 (2H, m), 7.01-6.93 (1H, m), 3.81-3.70 (1H, m), 3.58-3.48 (1H, m), 3.14-2.91 (2H, m), 2.92-2.77 (2H, m), 2.26-2.15 (1H, m), 2.01-1.77 (4H, m), 1.70-1.58 (1H, m).

### INTERMEDIATE A

### (R)-(5-Chloromethyl-isoxazol-3-yl)-cyclohexyl-phenyl-methanol

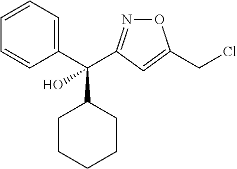

The title compound was obtained from (R)-cyclohexyl-hydroxy-phenyl-acetic acid as follows:

Step 1: 1,1′-Carbonyl diimidazole (25.0 g, 154 mmol) was added to a stirred suspension of (R)-cyclohexyl-hydroxy-phenyl-acetic acid (30.0 g, 128 mmol) in dry THF (600 mL). After stirring for 90 mins at room temperature, sodium borohydride (11.6 g, 307 mmol) was added portionwise over a period of 1 hour. The reaction mixture was then left to stir at room temperature overnight. The reaction was quenched by the addition of water (100 mL) then extracted with DCM. The combined organic phases were dried (MgSO₄), filtered and evaporated in vacuo to give a crude solid. Purification by silica gel chromatography (eluting with 0-5% methanol in DCM) gave (R)-1-cyclohexyl-1-phenyl-ethane-1,2-diol (20.7 g, 73%). ¹H NMR (400 MHz, CDCl₃): δ 7.41-7.33 (4H, m), 7.28-7.24 (1H, m), 3.99 (1H, d), 3.83 (1H, d), 2.68 (1H, br s), 1.86-1.80 (1H, m), 1.78-1.64 (3H, m), 1.63-1.57 (1H, m), 1.47-1.41 (1H, m), 1.27-0.94 (5H, m).

Step 2: A solution of oxalyl chloride (15.5 mL, 201 mmol) in dry DCM (900 mL) was cooled to −78° C. under a nitrogen atmosphere. A solution of DMSO (28.5 mL, 401 mmol) in DCM (25 mL) was added dropwise then the mixture stirred at −78° C. for 10 mins. A solution of (R)-1-cyclohexyl-1-phenyl-ethane-1,2-diol (29.5 g, 134 mmol) in DCM (250 mL) was added dropwise over the course of 1 hour giving a thick slurry. The internal temperature was allowed to reach −45° C. Triethylamine (92.8 mL, 669 mmol) was added dropwise and after complete addition the mixture was allowed to warm to room temperature. The mixture was washed with 1 N hydrochloric acid (500 mL×2), water (500 mL) and brine (500 mL) then dried (MgSO₄), filtered and evaporated to give an orange-coloured oil. This was dissolved in IMS (320 mL) and added portionwise to a preformed solution of hydroxylamine hydrochloride (14.0 g, 201 mmol) and sodium carbonate (21.3 g, 201 mmol) in water (210 mL). The resulting emulsion was stirred at room temperature overnight then partitioned between DCM and water. The organic layer was washed with water and brine, then dried (MgSO₄), filtered and evaporated in vacuo. Purification by silica gel chromatography (eluting with 0-15% EtOAc in cyclohexane) gave (R)-cyclohexyl-hydroxy-phenyl-acetaldehyde oxime (25.9 g, 83%). ¹H NMR (400 MHz, CDCl₃): δ 7.76 (1 H, s), 7.44-7.41 (2H, m), 7.37-7.33 (2H, m), 7.27-7.23 (1H, m), 7.22 (1H, br s), 3.34 (1 H, s), 1.90-1.60 (5H, m), 1.37-1.05 (6H, m).

Step 3: A solution of (R)-cyclohexyl-hydroxy-phenyl-acetaldehyde oxime (8 g, 34 mmol) and 2,6-lutidine (10 mL, 86 mmol) in DCM (150 mL) was cooled in an ice-bath. Trimethylsilyl trifluoromethanesulfonate (15.6 mL, 86 mmol) was added dropwise. The mixture was stirred for 10 minutes at 0° C. then allowed to warm to room temperature for 30 mins. The reaction was quenched by addition of water (50 mL). The organic phase was isolated by passage though a phase separation cartridge and evaporated in vacuo. Purification by silica gel chromatography (eluting with 10-20% EtOAc in cyclohexane) gave a mixture of mono and bis TMS-protected compounds. This was dissolved in methanol and left at room temperature overnight and evaporated in vacuo to give (R)-cyclohexyl-phenyl-trimethylsilanyloxy-acetaldehyde oxime (10 g, 96%). ¹H NMR (400 MHz, CDCl₃): δ 7.62 (1H, s), 7.32-7.28 (4H, m), 7.26-7.21 (1H, m), 7.11 (1H, s), 1.93-1.85 (2H, m), 1.76-1.71 (1H, m), 1.68-1.56 (2H, m), 1.49-1.42 (1H, m), 1.27-0.78 (5H, m), 0.11 (9H, m).

Step 4: A solution of (R)-cyclohexyl-phenyl-trimethylsilanyloxy-acetaldehyde oxime (6 g, 19.6 mmol) was formed in dry DCM (400 mL) and cooled to −78° C. Under reduced lighting, a solution of tert-butylhypochlorite (4.3 g, 39.3 mmol) in DCM (10 mL) was added dropwise. After 2 hours at −78° C. a solution of triethylamine (4.1 mL, 29.4 mmol) in DCM (10 mL) was added dropwise. After a further 10 mins at −78° C. the mixture was allowed to warm to 0° C. At this point, propargyl chloride (14.4 mL, 196 mmol) was added and the mixture was allowed to warm to room temperature overnight. The mixture was washed with brine (200 mL), dried (Na₂SO₄), filtered and evaporated. Purification by silica gel chromatography (eluting with 0-10% EtOAc in cyclohexane) gave crude 5-chloromethyl-3-((R)-cyclohexyl-phenyl-trimethylsilanyloxy-methyl)-isoxazole. This was re-dissolved in THF (100 mL), cooled in an ice-bath and a solution of tetrabutylammonium fluoride (19.6 mL of 1 M in THF) was added dropwise. This mixture was stirred for 30 mins at 0° C. then partitioned between ethyl acetate and water. The organic phase was dried (Na₂SO₄), filtered and evaporated in vacuo. Purification by silica gel chromatography (eluting with 0-20% EtOAc in cyclohexane) gave the title compound as a white solid (3.5 g, 58%). ¹H NMR (400 MHz, CDCl₃): δ 7.51 (2H, m), 7.32 (2H, m), 7.25-7.21 (1H, m), 6.29 (1H, s), 4.52 (2H, s), 2.80 (1H, s), 2.34-2.28 (1H, m), 1.81-1.76 (1H, m), 1.72-1.62 (3H, m), 1.36-1.02 (6H, m).

### INTERMEDIATE B

### (R)-(5-Bromomethyl-[1,3,4]oxadiazol-2-yl)-cyclohexyl-phenyl-methanol

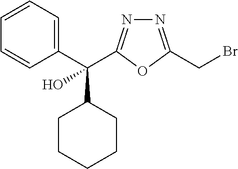

### Step 1: (R)-Cyclohexyl-hydroxy-phenyl-acetic acid hydrazide

A solution of (R)-cyclohexylmandelic acid (2.34 g) was dissolved in DCM (20 mL), treated with 1,1′-carbonyldiimidazole (1.95 g) and stirred at room temperature for 1 h. The reaction mixture was treated with hydrazine monohydrate (1.0 mL) and stirred for a further 30 minutes. The reaction mixture was diluted with DCM, washed with 1 N NaOH solution and brine, dried (MgSO₄), filtered and evaporated in vacuo to give the title compound as a white solid (2.0 g, 81%). LCMS (Method 2, 2.73 min). MH⁺=249.

### Step 2: Chloro-acetic acid N′—((R)-2-cyclohexyl-2-hydroxy-2-phenyl-acetyl)-hydrazide

A solution of the foregoing compound (1.0 g) was dissolved in DCM (20 mL) and treated at 0° C. with diisopropylethylamine (0.83 mL) and chloroacetyl chloride (0.39 ml). After warming to room temperature and stirring for 10 minutes, the reaction mixture was diluted with DCM, washed with water and brine, dried (MgSO₄), filtered and evaporated in vacuo to give the desired compound (1.1 g, 73%) as a white solid. LCMS (Method 2, 3.20 min). MH⁺=325.

### Step 3: (R)-(5-Chloromethyl-[1,3,4]oxadiazol-2-yl)-cyclohexyl-phenyl-methanol

A solution of the foregoing compound (170 mg), tosyl chloride (96 mg) and 1,2,2,6,6-pentamethylpiperidine (175 mg) in DCM (2 mL) was stirred at room temperature overnight. The reaction mixture was diluted with DCM, washed with NaHCO₃ solution (twice), brine, dried (MgSO₄), filtered and evaporated in vacuo. Purification by column chromatography (silica, 0-100% cyclohexane/ethyl acetate) gave the title compound as a white solid (105 mg, 63%). Data for the title compound: LCMS (Method 2, 3.79 min). MH⁺=307.

Step 4: A solution of the foregoing compound (4.66 g) and lithium bromide (6.6 g) in acetone (200 ml) was refluxed overnight. The reaction mixture was cooled, evaporated in vacuo and partitioned between water and ethyl acetate. The organic phase was separated, dried (MgSO₄), filtered and evaporated in vacuo. The resulting solid was redissolved in acetone (200 ml), treated with lithium bromide (6.6 g) and heated to reflux overnight. The reaction mixture was cooled, concentrated in vacuo and partitioned between water and ethyl acetate. The organic phase was separated, dried (MgSO₄), filtered and evaporated in vacuo to yield the title compound 4.65 g, 84%). Data for the title compound: LCMS (Method 2, 3.90 min). MH⁺=353. ¹H NMR δ (ppm) (CHCl₃-d): 7.60-7.53 (2H, m), 7.41-7.25 (3H, m), 4.49 (2H, s), 3.28 (1H, s), 2.33 (1H, s), 1.85-1.73 (1H, m), 1.68 (3H, s), 1.44-1.09 (6H, m).

### INTERMEDIATE C

### (5-Bromomethyl-isoxazol-3-yl)-diphenyl-methanol

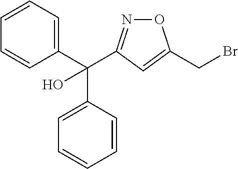

The title compound was obtained from methyl 5-methylisoxazole-3-carboxylate as follows:

Step 1. Phenylmagnesium Bromide (3 M solution in ether; 100 mL) was added dropwise to a solution of methyl 5-methylisoxazole-3-carboxylate (20.2 g) in anhydrous THF (300 mL) at −10° C. under a nitrogen atmosphere. The reaction mixture was stirred at −10° C. for 5 mins, then allowed to warm up to RT and left to stand for 18 hours. The reaction mixture was poured into cold 1 M HCl (300 mL) and extracted with ether. The combined organic extracts were washed with NaHCO₃, water, and brine, dried (MgSO₄), filtered and evaporated in vacuo to give (5-methyl-isoxazol-3-yl)-diphenyl-methanol (37.21 g, 98%) as a waxy solid. ¹H NMR (400 MHz, CDCl₃): δ 7.39-7.25 (m, 10H), 5.84 (s, 1H), 3.69 (s, 1 H), 2.38 (s, 3H).

Step 2. Dry 1,2-DCE (500 mL) was purged with argon for 15 mins. (5-Methyl-isoxazol-3-yl)-diphenyl-methanol (37.9 g) was added under nitrogen with stirring followed by NBS (28.0 g) and AIBN (4.7 g). The reaction mixture was stirred at 80° C. for 1 hour. Further NBS (28.0 g) and AIBN (4.7 g) was added to the reaction mixture and stirring continued at 80° C. for 3 hours. The reaction mixture was allowed to cool to RT, poured into 1M HCl (500 mL) and extracted with ether. The combined organic extracts were washed with NaHCO₃, water, and brine, dried (MgSO₄), filtered and evaporated in vacuo. Purification by silica gel chromatography eluting with 10-100% cyclohexane-DCM gave the title compound (26.0 g, 52%) as a pale yellow solid containing smaller amounts of unchanged starting material, and dibrominated- and tribrominated impurities. Data for the title compound: ¹H NMR (400 MHz, CDCl₃): δ 7.38-7.23 (m, 10H), 6.18 (s, 1H), 4.35 (s, 2H), 3.63 (s, 1H).

### EXAMPLE 1

### (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane chloride

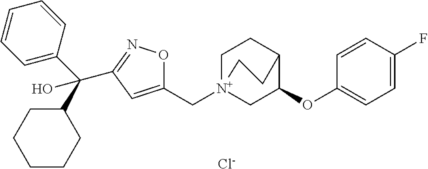

(R)-(5-Chloromethyl-isoxazol-3-yl)cyclohexyl-phenyl-methanol (Intermediate A) (1.74 g) and (R)-3-(4-fluoro-phenoxy)-1-aza-bicyclo[2.2.2]octane (Intermediate 2) (1.26 g) were mixed in acetonitrile (25 mL) and heated at 50° C. for 1 h. The resulting white solid was collected by filtration, washed with ethyl acetate and ether and dried in vacuo to yield the title compound (2.9 g). This was dissolved in boiling acetonitrile (125 ml) and allowed to cool slowly to room temperature whilst being stirred. The resulting crystals were collected by filtration and dried in vacuo to yield the title compound (2.4 g, 81%). Data for Example 1: ¹H NMR (400 MHz, DMSO-d6): δ 7.51-7.46 (m, 2H), 7.32 (t, 2H), 7.25-7.12 (m, 3H), 7.02-6.95 (m, 2H), 6.79 (s, 1H), 5.90 (s, 1H), 4.88 (s, 1H), 4.77 (s, 2H), 3.91 (dd, 1 H), 3.54-3.34 (m, 5H), 2.39 (s, 1H), 2.24-2.09 (m, 2H), 2.06-1.97 (m, 1H), 1.94-1.80 (m, 2H), 1.68 (d, 1H), 1.58 (d, 3H), 1.28-1.13 (m, 3H), 1.10-0.98 (m, 3H). LCMS (Method 1, 8.68 min). M⁺=491.

### EXAMPLE 2

### (R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane benzenesulfonate

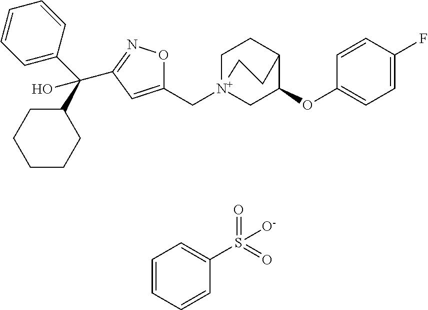

A solution of (R)-1-[3-((R)-cyclohexyl-hydroxy-phenyl-methyl)-isoxazol-5-ylmethyl]-3-(4-fluoro-phenoxy)-1-azonia-bicyclo[2.2.2]octane; chloride (Example 1) (2.0 g) was dissolved in DCM (20 ml) and stirred briskly with a solution of sodium benzenesulfonate (3.4 g) in water (20 ml). The organic layer was separated and stirred briskly again with a solution of sodium benzenesulfonate (3.4 g) in water (20 ml). The organic layer was dried (MgSO₄), filtered and evaporated in vacuo to give the title compound as a white foam. This was dissolved in boiling propan-2-ol (48 ml). The hot solution was filtered, and the filtrate was allowed to cool slowly to room temperature while being stirred. After 2 h, the mixture was cooled to 0° C., and the crystals were collected by filtration and dried in vacuo. The title compound (2.1 g) was obtained in 85% yield. ¹H NMR δ (ppm) (DMSO-d₆): 7.62-7.58 (2 H, m), 7.52-7.47 (2H, m), 7.35-7.26 (5H, m), 7.26-7.13 (3H, m), 7.02-6.95 (2H, m), 6.80 (1H, s), 5.89 (1H, s), 4.88 (1H, s), 4.75 (2H, s), 3.91 (1H, dd, J=13.17, 8.11 Hz), 3.58-3.35 (5H, m), 2.40 (1H, s), 2.25-1.95 (3H, m), 1.96-1.80 (2H, m), 1.69 (1H, d, J=10.55 Hz), 1.63-1.52 (3H, m), 1.29-0.96 (6H, m). LCMS (Method 1, 8.73 min). M⁺=491.

A sample of crystalline material was analysed by DSC, TGA, PXRD and DVS.

The melting temperature was determined by DSC at 10° C./min and found to have a sharp endothermic event with an onset temperature of 178° C. (±1° C.). Weight loss prior to melting was negligible by TGA. PXRD analysis showed the sample to be highly crystalline (see FIG. 1). DVS analysis produced a weight increase of 0.2% (% w/w) at 80% RH (±0.1%).

### EXAMPLE 3

### (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride

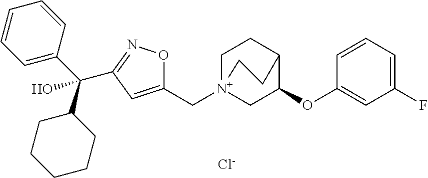

(R)-(5-Chloromethyl-isoxazol-3-yl)cyclohexyl-phenyl-methanol (Intermediate A) (3.00 g) and (R)-3-(3-fluoro-phenoxy)-1-aza-bicyclo[2.2.2]octane (Intermediate 1) (2.17 g) were mixed in acetonitrile (60 mL) and heated at 50° C. for 2 h. The reaction mixture was evaporated in vacuo and purified by silica gel chromatography (eluting with 1-15% methanol in DCM) to give the title compound as a white foam. This was dissolved in boiling acetonitrile (500 ml) and allowed to cool slowly to room temperature. The resulting white crystals were collected by filtration and dried in vacuo to give the title compound (3.9 g, 75%). ¹H NMR (400 MHz, DMSO-d6): δ 7.49 (dd, 2H), 7.40-7.29 (m, 3H), 7.25-7.20 (m, 1H), 6.93-6.79 (m, 4H), 5.90 (s, 1H), 4.96 (s, 1H), 4.77 (s, 2H), 3.95 (dd, 1 H), 3.49 (d, 4H), 2.43 (s, 1H), 2.26-2.10 (m, 2H), 2.07-1.98 (m, 1H), 1.95-1.82 (m, 2 H), 1.69 (d, 1H), 1.59 (s, 4H), 1.28-1.14 (m, 3H), 1.10-0.98 (m, 3H). LCMS (Method 1, 8.70 min). M⁺=491.

A sample of crystalline material was analysed by DSC, XRPD and GVS.

The melting temperature was determined by DSC and found to have a broad endothermic event (melt) onset approximately 134° C. (±2° C.). XRRD analysis showed the sample to be crystalline (see FIG. 2). GVS analysis produced a mass increase of approximately 5% 1^{st} cycle and 6.5% 2^{nd} cycle at 80% RH.

### EXAMPLE 4

### (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate

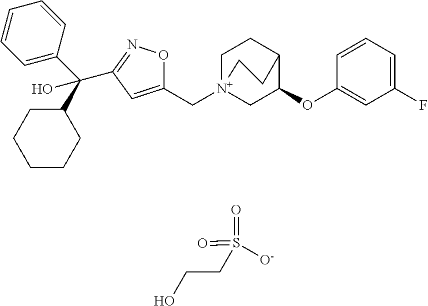

A solution of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane; chloride (Example 3) (3.2 g) in warm DCM (50 ml) and methanol (0.5 ml) was stirred briskly and treated with a solution of ammonium isethionate (5 g) in water (20 ml). The reaction mixture was stirred at room temperature for 1 h, then cooled to 0° C. and stirred for 0.5 h. The resulting white precipitate was collected by filtration and washed with water and ether and dried in vacuo. The precipitate was dissolved in boiling acetonitrile (172 ml). The resulting solution was filtered whilst hot, and allowed to cool slowly to room temperature whilst being stirred. After 2 h, the resulting white crystals were collected by filtration and dried in vacuo to give the title compound (3.07 g, 82%). ¹H NMR δ (ppm) (DMSO-d₆): 7.47-7.42 (2H, m), 7.35-7.25 (3H, m), 7.21-7.13 (1H, m), 6.81 (4H, d, J=43.75 Hz), 5.84 (1H, s), 4.92 (1H, s), 4.70 (2H, s), 4.40 (1H, t, J=5.72 Hz), 3.90 (1H, dd, J=13.18, 8.10 Hz), 3.58 (2H, td, J=6.74, 5.72 Hz), 3.48-3.29 (5H, m), 2.56 (2H, t, J=6.74 Hz), 2.39 (1H, s), 2.21-2.04 (2 H, m), 2.03-1.94 (1H, m), 1.93-1.77 (2H, m), 1.64 (1H, d, J=10.36 Hz), 1.54 (3H, d, J=9.07 Hz), 1.24-1.10 (3H, m), 1.10-0.93 (3H, m). LCMS (Method 1, 8.72 min). M⁺=491.

A sample of crystalline material was analysed by DSC, XRPD and GVS.

The melting temperature was determined by DSC and found to have a sharp melt onset at approximately 214° C. (±2° C.). XRPD analysis showed the sample to be crystalline (see FIG. 3). GVS analysis produced no mass increase at 80% RH.

### EXAMPLE 5

### (R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane bromide

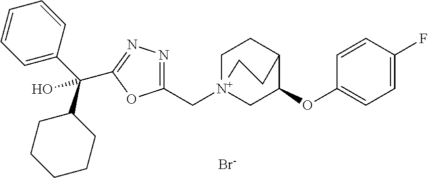

A solution of (R)-(5-bromomethyl-[1,3,4]oxadiazol-2-yl)-cyclohexyl-phenyl-methanol (Intermediate B) (2.93 g) and (R)-3-(4-fluoro-phenoxy)-1-aza-bicyclo[2.2.2]octane (Intermediate 2) (1.8 g) in acetonitrile (60 ml) was heated at 50° C. overnight. The reaction mixture was evaporated in vacuo and triturated with ether to yield the title compound (4.7 g), which was recrystallized from boiling ethyl acetate. ¹H NMR (400 MHz, DMSO-d6): δ 7.44-7.39 (m, 2H), 7.34-7.21 (m, 3H), 7.16-7.09 (m, 2H), 6.97-6.90 (m, 2H), 6.39 (s, 1H), 4.92 (s, 2H), 4.82 (s, 1H), 3.97-3.87 (m, 1H), 3.59-3.37 (m, 5H), 2.38 (s, 1H), 2.22 (t, 1H), 2.11 (s, 1H), 2.00 (s, 1H), 1.84 (s, 2H), 1.66 (s, 2H), 1.57 (t, 2H), 1.32 (d, 1H), 1.23-1.00 (m, 3H), 1.03-0.88 (m, 2H). LCMS (Method 1, 8.29 min). M⁺=492.

A sample of crystalline material was analysed by DSC, XRPD and GVS.

The melting temperature was determined by DSC and a double endothermic event was observed. The melt onset was assumed to be approximately 169° C. (±2° C.). XRPD analysis showed the sample to be crystalline (see FIG. 4). GVS analysis produced a mass increase of approximately 0.8% at 80% RH.

### EXAMPLE 6

### (R)-3-(3-Fluoro-phenylsulfanyl)-1-[3-(hydroxy-diphenyl-methyl)-isoxazol-5-ylmethyl]-1-azonia-bicyclo[2.2.2]octane bromide

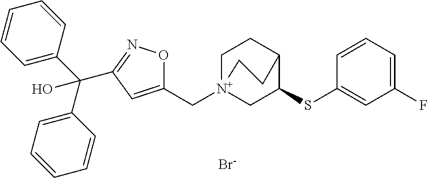

A solution of (5-bromomethyl-isoxazol-3-yl)-diphenyl-methanol (Intermediate C) (1.1 g of an approximately 40% pure sample) and (R)-3-(3-fluoro-phenylsulfanyl)-1-aza-bicyclo[2.2.2]octane (Intermediate 4) (218 mg) in acetonitrile (10 ml) was stirred at room temperature for 1 h. The resulting precipitate was collected by filtration and dried in vacuo. This was dissolved in boiling acetonitrile (130 ml), filtered whilst hot, and allowed to cool slowly to room temperature whilst stirring. The resulting crystals were collected by filtration and dried in vacuo to yield the title compound (312 mg, 51%). ¹H NMR δ (ppm) (400 MHz, CH₃OH-d₄): 7.40-7.22 (13H, m), 7.09-7.03 (1H, m), 6.83 (1H, s), 4.71 (2H, s), 4.07-3.98 (2H, m), 3.69-3.38 (5H, m), 2.50-2.39 (1H, m), 2.29-2.25 (1H, m), 2.24-2.14 (1H, m), 2.18-1.93 (2H, m). LCMS (Method 1, 8.36 min). M⁺=501.19.

### EXAMPLE 7

### (R)-3-(3-Fluoro-4-methyl-phenoxy)-1-[3-(hydroxy-diphenyl-methyl)-isoxazol-5-ylmethyl]-1-azonia-bicyclo[2.2.2]octane bromide

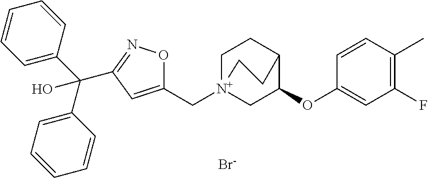

A solution of (5-bromomethyl-isoxazol-3-yl)-diphenyl-methanol (Intermediate C) (4.7 g of an approximately 67% pure sample) and (R)-3-(3-fluoro-4-methyl-phenoxy)-1-aza-bicyclo[2.2.2]octane (Intermediate 3) (2 g) in acetonitrile (50 ml) was heated at 50° C. for 1.5 h. The reaction mixture was cooled and the solid was collected by filtration and washed with ethyl acetate and ether and dried in vacuo to give the title compound (4.36 g, 88%). This was dissolved in boiling propan-2-ol (760 ml), filtered whilst hot, and allowed to cool slowly to room temperature whilst stirring. The resulting crystals were collected by filtration and dried in vacuo to yield the title compound (3.72 g). ¹H NMR δ (ppm) (400 MHz, CH₃OH-d₄): 7.39-7.26 (10H, m), 7.16 (1H, t, J=8.63 Hz), 6.84 (1H, s), 6.75-6.66 (2H, m), 4.93-4.87 (1H, m), 4.79-4.70 (2H, m), 4.03-3.95 (1H, m), 3.67-3.48 (5H, m), 2.56-2.52 (1H, m), 2.40-2.31 (1H, m), 2.20-2.11 (4H, m), 2.10-1.93 (2H, m). LCMS (Method 1, 8.37 min). M⁺=499.20.

A sample of crystalline material was analysed by DSC, XRPD and GVS.

The melting temperature was determined by DSC and found to have a sharp melt onset at approximately 242° C. (±2° C.). XRPD analysis showed the sample to be crystalline (see FIG. 5). GVS analysis produced a mass increase of approximately 0.1% at 80% RH.

### EXAMPLE 8

### (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate

To a stirred suspension of (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride¹ (155.83 g) and DCM (2380 mL) in a 5 L flask equipped with an overhead stirrer was added MeOH (23.8 mL) in one portion. After stirring for a few minutes a solution formed. To the stirred solution of the chloride salt was added a solution of isethionic acid, ammonium salt (61.60 g) in water (945 mL) over 5 minutes. The resulting two-phase reaction mixture was stirred vigorously and after a few minutes some seed crystals of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate were added. A few more were added after a further 35 minutes of stirring. Traces of solid formation were observed around the sides of the flask. It was stirred at room temperature for a further 2.5 hours and a dense precipitate began to form. Examination of a small aliquot of the reaction mixture under a microscope showed crystalline material. The stirred reaction mixture was cooled in an ice bath (with internal temperature 4° C. for 35 minutes). The solid became more granular. The solid was collected by filtration and washed with cold water (total volume 3.1 L in 400-60 mL portions) and then with ether (5×500 mL). It was sucked dry in air and then dried in vacuo at 40° C. overnight and then for a further 6 hours to give the product as a white crystalline solid (152.48 g). LC-MS (Method 2): Rₜ 8.91 min, m/z 491 [M]⁺. Purity >99%.

The product (152.48 g) was then dissolved with stirring in refluxing IMS (2.8 L) and the hot solution was filtered. This solution was kept hot and stirred in a 10 L heated jacket reactor whilst the remaining material (151.64 g) was dissolved in refluxing IMS (2.8 L) and then filtered hot. The two solutions were combined in a 10 L heated jacket reactor and stirred and refluxed. A small amount of material had started to crystallise out, so further IMS (350 mL) was added until a solution formed. The stirred solution (stirring speed 88-89 rpm) was gradually allowed to cool [78° C. (reflux temperature) to 76.5° C. (internal temperature) over about 1 h and then 76.5-20° C. (internal temperature) over 4.5 hours and then stirred at 20° C. overnight]. Seed crystals were added to the stirred solution at 77° C., 69° C. and 59° C. Solid material had begun to crystallise out at base of reactor. More crystallisation was observed over the next few minutes as the mixture cooled down further. After stirring overnight the solid was collected by filtration, washed with cold IMS (˜300 ml) and dried by suction in air (for 2.5 hours) and then in vacuo at 40° C. overnight to give crystalline (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (274.48 g). LC-MS (Method 2): Rₜ 8.84 min, m/z 491 [M]⁺. Purity >99%. ¹A preparation of (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride is described in Example 3 and in WO 2008/099186.

A sample of crystalline material was analysed by XRPD, GVS and DSC. The melting temperature as determined by DSC was found to be 213° C. (onset) (±2° C.). GVS determination gave a weight increase of 0.15% at 80% RH (±0.3%). An XRPD spectrum is presented in FIG. 6.

### Biological Activity of Muscarinic Antagonists

The inhibitory effects of compounds of the muscarinic antagonists were determined by a Muscarinic Receptor Radioligand Binding Assay.

Recombinant human M3 receptor was expressed in CHO-K1 cells. Cell membranes were prepared and binding of [3H]-N-methyl scopolamine ([3H]-NMS) and compounds was assessed by a scintillation proximity assay (SPA). The incubation time was 16 hours at ambient temperature in the presence of 1% (v/v) DMSO. The assay was performed in white 96 well clear-bottomed NBS plates (Corning). Prior to the assay, the CHO cell membranes containing M3 receptor were coated onto SPA WGA (Wheat germ agglutinin) beads (GE Healthcare). Non specific binding was determined in the presence of 1 μM Atropine.

Radioactivity was measured on a Microbeta scintillation counter (PerkinElmer) using a 3H protocol with a 2 minutes per well read time. Compound inhibition of [3H]-NMS binding was determined typically using concentrations in the range 0.03 nM to 1 μM and expressed as percent inhibition relative to the plate specific radioligand binding for the plate. Concentration dependent inhibition of [3H]-NMS binding by compounds was expressed as pIC50.

All compounds tested exhibited potencies (as Ki values) in the M3 binding assay of less than 5 nM. In particular, Example 1 exhibited a Ki of 0.80 nM, Example 3 exhibited a Ki of 0.66 nM, Example 5 exhibited a Ki of 0.70 nM, Example 6 exhibited a Ki of 0.15 nM and Example 7 exhibited a Ki value of 0.40 nM in the M3 binding assay.

### Protocols for Combination Experiments

1. Evaluation of Compound Activity on Isolated Tracheal Rings from Guinea-Pig Preconstricted with Methacholine.

The following protocol may be used to evaluate the effects of a muscarinic M3 receptor antagonist according to the present invention in combination with a CCR1 antagonist.

The following protocol may be used to evaluate the effects of muscarinic M3 receptor antagonists according to the present invention in combination with budesonide.

Male albino Dunkin Hartley guinea-pigs (300-350 g) are killed by cervical dislocation and the trachea excised. Adherent connective tissue is removed and the trachea cut into ring segments (2-3 mm wide). These are suspended in 10 ml organ baths containing a modified Krebs solution composition (mM): NaCl 117.56, KCl 5.36, NaH₂PO₄ 1.15, MgSO₄ 1.18, glucose 11.10, NaHCO₃ 25.00 and CaCl₂ 2.55. This is maintained at 37° C. and continually gassed with 5% CO₂ in O₂, Indomethacin (2.8 μM), corticosterone (10 μM), ascorbate (1 mM), CGP20712A (1 μM) and phentolamine (3 μM) are added to the Krebs solution: indomethacin to prevent development of smooth muscle tone due to the synthesis of cyclooxygenase products, corticosterone to inhibit the uptake 2 process, ascorbate to prevent catecholamine oxidation and CGP20712A and phentolamine to avoid any complicating effects of β1- and α-adrenoceptor activation respectively. The tracheal rings are suspended between two stainless steel hooks, one attached to an isometric force transducer and the other to a stationary support in the organ bath. Changes in isometric force are recorded.

Acetyl-β-methylcholine chloride (Methacholine), Indomethacin, Corticosterone-21-acetate, Phentolamine hydrochloride, Ascorbic acid, and CGP20712A methanesulphate may be obtained from the Sigma chemical company. Indomethacin may be dissolved in 10% w/v Na₂CO₃, corticosterone 21-acetate in ethanol and other compounds in DMSO. Muscarinic antagonists and Budesonide may be diluted in Krebs prior to adding to tissues and the level of DMSO in the bath <0.1%.

At the beginning of each experiment a force of 1.0 g·wt. is applied to the tissues and this is reinstated over a 30 min equilibration period until it remained steady. Tissues are then exposed to 1 μM of the muscarinic agonist, methacholine, to assess tissue viability. Tissues are washed by exchanging the bathing Krebs solution three times. After 30 minutes the tissues are precontracted with 1 μM methacholine. When the contraction reaches a plateau, 100 nM Budesonide, 10 nM Muscarinic antagonist or a combination of the two is added to the bathing media and left for 60 minutes.

Data may be collected using the ADInstruments Chart5 for windows software, the tension generated may be measured before addition of methacholine and after its response reaches a plateau. The response to the muscarinic antagonist and/or Budesonide may be measured at 10 minute intervals following their addition. All responses may be expressed as percentage inhibition of the methacholine-induced contraction.

### 2. Inflammatory Cell Influx Experiment in LPS-Challenged Rats

The following protocol may be used to evaluate the effects of a muscarinic M3 receptor antagonist according to the present invention in combination with a CCR1 antagonist.

The following protocol may be used to evaluate the effects of muscarinic M3 receptor antagonists according to the present invention, in combination with CCR1 anatgonists.

The effect of a CCR1 receptor antagonist and a muscarinic antagonist according to the invention, and their combination, on inflammatory cell influx can be assayed by monitoring the effect on total cell number in broncholalveolar lavage (BAL) fluid of rats challenged intra-tracheally (i.t.) with Lipopolyaccharide (LPS) [N=10 rats per treatment group].

### Methodology

LPS instillation: Rats are anaesthetized with Efrane and put in a supine position, head up, on a board tilted at 30°. LPS (Lipopolysaccharide B.E. coli 026:B6) (2.5 μg/ml) is dissolved in saline (0.9% NaCl), or saline alone (negative control) in a volume of 200 μl and administered i.t. using a modified metal cannula. Rats remain in this position until regaining consciousness.

Preparation of solutions: CCR1 anatgonists are dissolved in 0.9% NaCl solution to a final concentration of 0.001 to 0.100 mg. Muscarinic antagonists are dissolved in 0.9% NaCl solution to an appropriate final concentration of 0.001 to 1.0 mg/ml. CCR1 antagonist, Muscarinic antagonist or mixed s are made by dissolving CCR1 antagonist in Muscarinic antagonist suspensions, giving a final concentration of 001 to 0.100 CCR1 antagonist/ml and 001 to 1.0 mg Muscarinic antagonist/ml.

Treatments: Animals were intratracheally instilled with solutions (1 ml/kg) of Muscarinic antagonist/CCR1 antagonist (0.002/001 to 0.100 mg/kg), or of Muscarinic antagonist (001 to 1.0 mg/kg) alone, or CCR1 antagonist (001 to 0.100 mg/kg) alone, or with saline (negative and positive control animals). The treatments were carried out under light anaesthesia (Efrane) to secure that the solution reached the lungs. The drugs were administrated 30 min before the LPS instillation.

Termination: 4 hours after the LPS challenge, rats are intraperitoneally injected with the mixture (0.3 ml) of pentobarbital (60 mg/ml, Apoteksbolaget, Sweden) and PBS (1:1) for 1-2 min.

Bronchoalveolar lavage: After termination, BAL is performed twice with PBS. The BAL fluid is centrifuged and the cell pellet was resuspended in PBS. The total numbers of BAL cells is counted in a SYSMEX cell counter.

1. A pharmaceutical product comprising, in combination, a first active ingredient which is a muscarinic antagonist selected from:
(R)-1-[5-((R)-Cyclohexyl-hydroxy-phenyl-methyl)-[1,3,4]oxadiazol-2-ylmethyl]-3-(4-fluoro-phenoxy)-1-azonia-bicyclo[2.2.2]octane X;
(R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane X;
(R)-3-(3-Fluoro-4-methyl-phenoxy)-1-[3-(hydroxy-diphenyl-methyl)-isoxazol-5-ylmethyl]-1-azonia-bicyclo[2.2.2]octane X;
(R)-3-(3-Fluoro-phenylsulfanyl)-1-[3-(hydroxy-diphenyl-methyl)-isoxazol-5-ylmethyl]-1-azonia-bicyclo[2.2.2]octane X;
(R)-1-[3-((R)-Cyclohexyl-hydroxy-phenyl-methyl)-isoxazol-5-ylmethyl]-3-(4-fluoro-phenoxy)-1-azonia-bicyclo[2.2.2]octane X;
wherein X represents a pharmaceutically acceptable anion of a mono or polyvalent acid, and a second active ingredient which is selected from
i) a phosphodiesterase inhibitor,
ii) a modulator of chemokine receptor function
iii) an inhibitor of kinase function,
iv) a protease inhibitor,
v) a steroidal glucocorticoid receptor agonist,
vi) a non-steroidal glucocorticoid receptor agonist, and
vii) a purinoceptor antagonist.

2. A product according to claim 1 wherein the first active ingredient is a muscarinic antagonist, which is a bromide salt.

3. A product according to claim 1 or claim 2, wherein the second active ingredient is a CCR1 antagonist.

4. A product according to claim 3 wherein the second active ingredient is a CCR1 antagonist, selected from:
N-{2-[((2S)-3-{[1-(4-chlorobenzyl)piperidin-4-yl]amino}-2-hydroxy-2-methylpropyl)oxy]-4-hydroxyphenyl}acetamide;
N-{5-chloro-2-[((2S)-3-{[1-(4-chlorobenzyl)piperidin-4-yl]amino}-2-hydroxy-2-methylpropyl)oxy]-4-hydroxyphenyl}acetamide;
2-{2-chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid;
or pharmaceutically acceptable salts thereof.

5. A product according to claim 4, wherein the second active ingredient is N-{2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof.

6. A product according to claim 4, wherein the second active ingredient is N-{5-chloro-2-[((2S)-3-{[1-(4-chlorobenzyl)piperidin-4-yl]amino}-2-hydroxy-2-methylpropyl)oxy]-4-hydroxyphenyl}acetamide or a pharmaceutically acceptable salt thereof.

7. A product according to claim 4, wherein the second active ingredient is 2-{2-chloro-5-{[(2S)-3-(5-chloro-1′H, 3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof.

8. A product according to claim 1 or claim 2, wherein the second active ingredient is a steroidal glucocorticoid receptor agonist.

9. Use of a product according to any one of claims 1 to 8 in the manufacture of a medicament for the treatment of a respiratory disease.

10. Use according to claim 9, wherein the respiratory disease is chronic obstructive pulmonary disease.

11. A method of treating a respiratory disease, which method comprises simultaneously, sequentially or separately administering:
(a) a (therapeutically effective) dose of a first active ingredient which is a muscarinic receptor antagonist as defined in claim 1 or claim 2; and
(b) a (therapeutically effective) dose of a second active ingredient as defined in claim 1; to a patient in need thereof.

12. A kit comprising a preparation of a first active ingredient which is a muscarinic receptor antagonist as defined in claim 1 or claim 2, and a preparation of a second active ingredient as defined in claim 1 and optionally instructions for the simultaneous, sequential or separate administration of the preparations to a patient in need thereof.

13. A pharmaceutical composition comprising, in admixture, a first active ingredient which is a muscarinic receptor antagonist as defined in claim 1 or claim 2 and a second active ingredient as defined in claim 1.
